(19) United States
(12) Patent Application Publication
(10) Pub. No.: US 2026/0144696 A1
(43) Pub. Date: May 28, 2026

(54) THERAPEUTIC DEVICE AND METHOD FOR USE IN THE TREATMENT OF SPASTIC HEMIPARESIS OF THE HAND

(71) Applicant: Theranetic Medical, Inc., Indianapolis, IN (US)

(72) Inventors: Shane M. Bauer, Indianapolis, IN (US); Supriya R. Ganti, Hermosa Beach, CA (US); Madelyn R. Geobel, Manchester Center, VT (US); Ross A. Gortner, III, South Strafford, VT (US); Harry P. Leiter, Cold Spring, NY (US); Daniel V. Will, III, Elgin, SC (US)

(21) Appl. No.: 19/456,513

(22) Filed: Jan. 22, 2026

(51) Int. Cl. A61H 1/02; A61F 5/058

(52) U.S. Cl. CPC A61H 1/0288; A61F 5/05866

(57) ABSTRACT

A therapeutic device for use in the treatment of spastic hemiparesis of the hand includes a splint and an install component positioned on a proximal end of the splint. The install component includes a generally conically-shaped guide body that a patient uses to guide advancement of the patient's hand onto the splint. The splint includes a pivoting finger support flange that extends the fingers of the patient. A method of using a therapeutic device in the treatment of spastic hemiparesis of the hand is also disclosed.

### TECHNICAL FIELD

The present disclosure relates generally to therapeutic devices and methods and, more particularly, to therapeutic devices and methods for use in the treatment of spastic hemiparesis of the hand.

### BACKGROUND

Spastic hemiparesis of the hand is a stiffness (spasticity) and weakness of a patient's hand. It often causes the patients to experience an uncontrollable clenched fist in which the fingers curl inwardly and, in some cases, the thumb is tucked within the fingers. Spastic hemiparesis often results from brain damage, commonly seen in cerebral palsy, stroke, or brain trauma patients in which faulty nerve signals cause the muscles of the hand to contract involuntarily. This leads to limited movement and reduced fine motor skills. Moreover, contracted muscles are shorter than uncontracted muscles and, if left untreated, patients are at risk of contracture of the hand where the hand muscles become permanently shortened.

Typical treatments for spastic hemiparesis of the hand may include occupational and physical therapy, splinting, stretching, and/or surgery. In the case of splinting, while some current devices have been shown to be effective, they are often very expensive or very difficult to put on the patient or both. Moreover, many existing splint devices do not provide for the ability to adjust the degree of stretch of the patient's fingers (for example, some devices only allow for a fully flat extension of the fingers). Yet further, existing splint devices do not emphasize the stretch of the thumb and the carpometacarpal (CMC) joint separately, but instead utilize the same unadjustable range for every finger (and thus do not reflect the differences of forces between them). In addition, many existing splint devices cannot be installed by the patient without assistance from a care provider (i.e., the patient cannot install the splint device by himself or herself).

### SUMMARY

According to one aspect of the disclosure, a therapeutic device for use in the treatment of spastic hemiparesis of the hand includes a splint and an install component. The splint has a grip that includes a planar thumb side and an opposite planar palm side with a rounded surface connecting the thumb side to the palm side. The splint also includes a thumb support extending medially out of the thumb side of the grip, and a finger support flange pivotally coupled to the grip. The install component is used to guide a hand of a patient onto the splint. The install component is removably secured to a proximal end of the splint and includes a rounded proximal tip and a generally conically-shaped guide body extending distally away from the tip. The conically-shaped guide body tapers outwardly in both the mediolateral direction and the anteroposterior direction from its proximal end to its distal end.

In an embodiment, the therapeutic device includes a base having a platform and a post extending upwardly from the base. The post is sized and shaped to be received into a bore formed in the grip so as to allow the grip to be removably secured to the base.

The thumb support may include an arcuate-shaped support surface.

In an embodiment, the arcuate-shaped support surface is curved downwardly from its proximal end to its distal end.

In an embodiment, the arcuate-shaped support surface is curved in the anteroposterior direction.

In an embodiment, the thumb support is upwardly and downwardly adjustable relative to the grip.

In an embodiment, the finger support flange is movable between an extended position and a retracted position. The lateral side of the finger support flange is aligned with the palm side of the grip when the finger support flange is positioned in its extended position, whereas the lateral side of the finger support flange is positioned perpendicular to the palm side of the grip when the finger support flange is positioned in its retracted position.

The therapeutic device may also include a deployment tool having an elongated key and an elongated cam. The elongated key is sized and shaped to be received into an elongated slot formed in the grip, whereas the elongated cam is configured to engage a medial side of the finger support flange when the key is positioned in the slot of the grip so as to move the finger support flange from its retracted position to its extended position.

In an embodiment, the elongated cam includes a cam surface which tapers downwardly from its proximal end to its distal end, and a planar surface opposite its cam surface. The elongated key includes planar medial and lateral surfaces. The planar surface of the elongated cam is perpendicular to the medial and lateral surfaces of the elongated key.

In an embodiment, the finger support flange is movable between an extended position and a retracted position. A lateral side of the finger support flange is positioned perpendicular to the palm side of the grip when the finger support flange is positioned in its retracted position, whereas the lateral side of the finger support flange defines an obtuse angle with the palm side of the grip when the finger support flange is positioned in its extended position.

According to another aspect, a method of using a therapeutic device in the treatment of spastic hemiparesis of the hand includes positioning an install component on a proximal end of a splint, and thereafter advancing a patient's hand distally along a conically-shaped guide body of the install component and onto a grip of the splint, the conically-shaped guide body tapering outwardly in both the mediolateral direction and the anteroposterior direction from its proximal end to its distal end. The method also includes pivoting, with the patient's hand positioned on the splint, a finger support flange of the splint from a retracted position to an extended position so as to extend one or more fingers of the patient's hand.

In an embodiment, during advancement of the patient's hand onto the grip of the splint, a thumb of the patient's hand is advanced into contact with a thumb side of the grip, and a palm of the patient's hand is advanced into contact with an opposite palm side of the grip.

In an embodiment, during advancement of the patient's hand onto the grip, a thumb of the patient's hand is advanced onto a thumb support extending medially out of the grip.

The method may also include positioning the grip on a base prior to advancement of the patient's hand onto the grip of the splint.

The method may also include removing the grip of the splint from the base prior to pivoting the finger support flange.

Pivoting the finger support flange may include inserting a deployment tool into the grip so as to pivot the finger support flange from its retracted position to its extended position.

Inserting the deployment tool into the grip may include inserting an elongated key of the deployment tool into the grip so as to cause an elongated cam of the deployment tool to pivot the finger support flange from its retracted position to its extended position.

According to another aspect, a splint for use in the treatment of spastic hemiparesis of the hand includes a grip having a planar thumb side and an opposite planar palm side with a rounded surface connecting the thumb side to the palm side. The splint also includes a thumb support extending medially out of the thumb side of the grip, with the thumb support having an arcuate-shaped support surface. The splint also includes a finger support flange pivotally coupled to the grip.

In an embodiment, the arcuate-shaped support surface is curved downwardly from its proximal end to its distal end.

In an embodiment, the arcuate-shaped support surface is curved in the anteroposterior direction.

In an embodiment, the thumb support is upwardly and downwardly adjustable relative to the grip.

In an embodiment, the finger support flange is movable between an extended position and a retracted position. The lateral side of the finger support flange is aligned with the palm side of the grip when the finger support flange is positioned in its extended position, whereas the lateral side of the finger support flange is positioned perpendicular to the palm side of the grip when the finger support flange is positioned in its retracted position.

In an embodiment, the finger support flange is movable between an extended position and a retracted position. A lateral side of the finger support flange is positioned perpendicular to the palm side of the grip when the finger support flange is positioned in its retracted position, whereas the lateral side of the finger support flange defines an obtuse angle with the palm side of the grip when the finger support flange is positioned in its extended position.

### BRIEF DESCRIPTION OF THE DRAWINGS

The detailed description particularly refers to the following figures, in which:

FIG. 1 is perspective view of a therapeutic device for use in the treatment of spastic hemiparesis of the hand;

FIG. 2 is view similar to FIG. 1, but showing the install component removed from the splint;

FIG. 3 is perspective view of a deployment tool for use with the splint of the therapeutic device of FIG. 1;

FIG. 4 is side elevation view of the deployment tool of FIG. 3;

FIG. 5 is a perspective view showing the deployment tool of FIG. 3 being used to pivot the splint's finger support flange relative to its grip;

FIG. 6 is a perspective view showing the grip therapeutic device removed from the device's base;

FIG. 7 is a perspective view showing the finger support flange in its extended position;

FIGS. 8 and 9 show a patient's hand being advanced along the install component toward the grip of the therapeutic device;

FIGS. 10 and 11 show the patient's hand fully advanced onto the grip of the therapeutic device;

FIG. 12 shows the deployment tool being used to move the finger support flange along with the patient's fingers; and

FIG. 13 shows the finger support flange of the splint in its extended position thereby having extended the patient's fingers.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the devices and tools described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

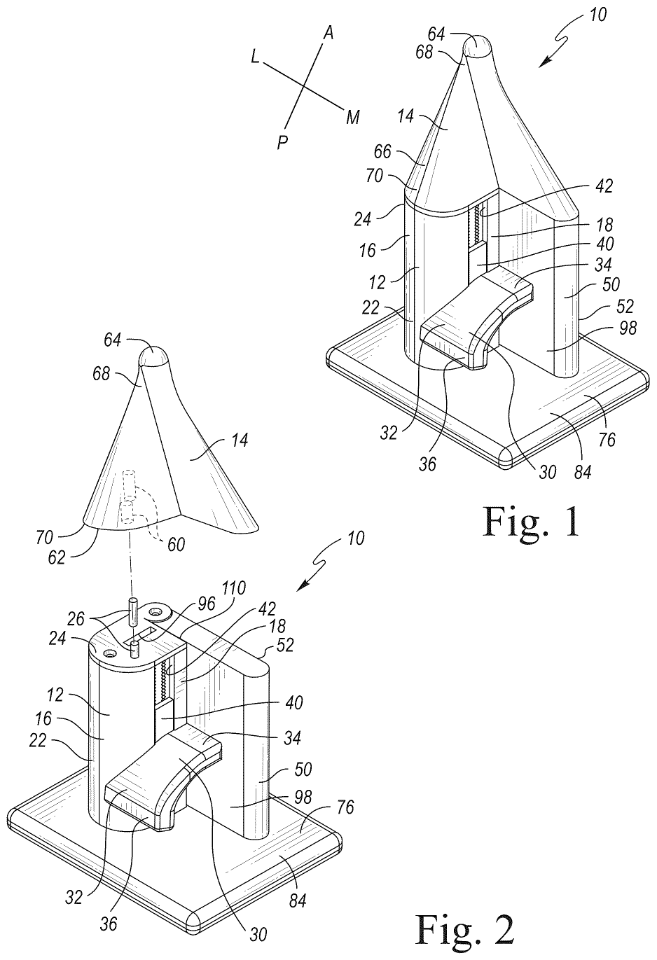

Referring to FIGS. 1 and 2, a therapeutic device 10 for use in the treatment of spastic hemiparesis of the hand is shown. As will be described below in more detail, the therapeutic device 10 may be used by a patient to stretch (i.e., extend) the patient's fingers and thumb as part of a regular therapeutic treatment process. In most cases, the patient can don (i.e., put on) the device's grip without the assistance of a care provider. The therapeutic device 10 may be provided in both righthand and lefthand configurations. Moreover, the therapeutic device 10 may be provided in multiple different sizes to fit the needs of patients across a range of hand sizes. The components of the therapeutic device 10 may be constructed of any suitable material, including polymers. In an illustrative embodiment, such polymer components are fabricated using additive manufacturing techniques (e.g., 3D printing).

The therapeutic device 10 includes a splint 12 and an install component 14. As will be described in more detail below, the install component 14 is used to guide the patient's hand onto the splint 12. The splint 12 includes a grip 16 that is clasped by the user during use of the splint 12. The grip 16 includes a planar thumb side 18 and an opposite planar palm side 20. A rounded surface 22 connects the thumb side 18 to the palm side 20. When the grip 16 is engaged by a patient, the patient's thumb contacts the thumb side 18 of the grip 16 with the patient's palm contacting the grip's palm side 20. The interdigital web between the patient's thumb and index finger wraps around the rounded surface 22 connecting the two sides 18, 20.

The splint 12 also includes a thumb support 30. As can be seen in FIGS. 1 and 2, the thumb support 30 extends medially out of the thumb side 18 of the grip 16. The thumb support 30 includes an arcuate-shaped support surface 32 that is curved in the anteroposterior direction. As such, the support surface 32 curves downwardly from its proximal end 34 to its distal end 36. Such a curvature allows the thumb support 30 to support the entirety of the patient's thumb including stabilization and support of the carpometacarpal (CMC) joint near its distal end 36.

As can be seen in FIGS. 1 and 2, a slide rail 40 is secured to the lateral side of the thumb support 30. The slide rail 40 is captured in a slot 42 formed in the thumb side 18 of the grip 16. As such, the thumb support 30 is upwardly and downwardly adjustable relative to the grip 16. Such adjustability allows for variability in patient and anatomy and existing joint flexibility. An indexable stop (not shown) is positioned in the slot 42 below the slide rail 40. The position of the stop may be adjusted to limit the travel of the slide rail 40—and hence the thumb support 30—at a given lower limit.

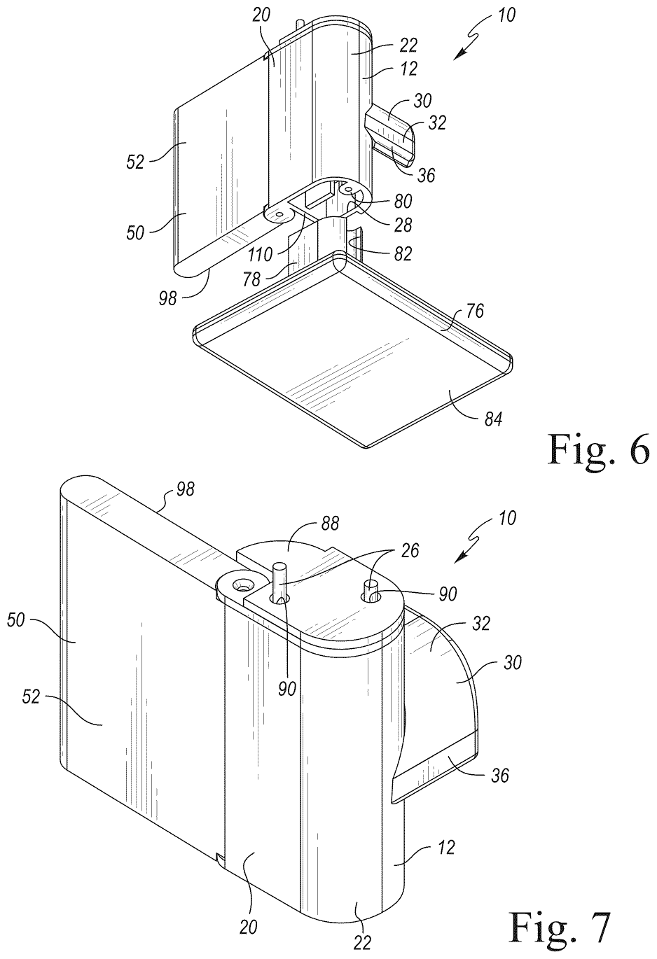

The splint 12 also includes a finger support flange 50 pivotally coupled to the grip 16. Although the finger support flange 50 is herein described as being coupled to the grip 16 via a pivot joint, it should be appreciated that other mechanical joints may be used to couple the articulated finger support flange 50 to the grip 16. The finger support flange 50 is movable between an extended position (e.g., as shown in FIGS. 6 and 7) and a retracted position (e.g., as shown in FIGS. 1 and 2). As can be seen in FIGS. 6 and 7, a lateral side 52 of the finger support flange 50 is aligned with (i.e., is parallel with) the palm side 20 of the grip 16 when the finger support flange 50 is positioned in its extended position, whereas the lateral side 52 of the finger support flange 50 is positioned perpendicular to the palm side 20 of the grip 16 when the finger support flange 50 is positioned in its retracted position. In some embodiments, the therapeutic device 10 may be operated to assist patients who are not capable of full extension of their fingers. In such cases, when positioned in its extended position, the lateral side 52 of the finger support flange 50 defines an obtuse angle with the palm side 20 of the grip 16 when the finger support flange 50 is positioned in its extended position. In other words, the finger support flange 50 is not extended all the way to a position in which its lateral side 52 is aligned with the palm side 20 of the grip 16. An example of the finger support flange 50 positioned in such a position is shown in FIG. 5.

As alluded to above, the install component 14 is used to guide the hand of a patient onto the splint 12. To do so, the install component 14 is removably secured to a proximal end 24 of the splint 12. As can be seen in FIG. 2, a pair of elongated mounting pins 26 extend out of the splint's proximal end 24. The pins 26 are received into a corresponding pair of blind bores 60 formed in the distal end 62 of the install component 14. The pins 26 not only couple the install component 14 to the splint 12, but they also maintain the install component 14 in proper alignment on the splint 12.

The install component 14 has a rounded proximal tip 64. A generally conically-shaped guide body 66 extends distally away from the tip 64. As can be seen in FIGS. 1 and 2, the conically-shaped guide body 66 tapers outwardly in both the mediolateral direction and the anteroposterior direction from its proximal end 68 to its distal end 70. Moreover, as can also be seen in FIGS. 1 and 2, the distal end 70 of the conically-shaped guide body 66 conforms to the size and shape of the proximal end 24 of the splint 12 and thus blends smoothly into the thumb side 18, the palm side 20, and the rounded surface 22 of the splint's grip 16. As will be described in more detail below, such a configuration allows the patient's hand to be spread during advancement along the conically-shaped guide body 66 and onto the grip 16.

As can be seen in FIGS. 1, 2, and 6, the therapeutic device 10 also includes a base 76. The base 76 includes a platform 84 that, in the illustrative embodiment described herein, is rectangular shaped with opposite planar surfaces. A post 78 extends upwardly from the base 76. The post 78 is sized and shaped to be received into a bore 28 formed in the grip 16 so as to allow the grip 16 to be removably secured to the base 76. As can be seen in FIG. 6, a rounded alignment key 80 is positioned in the grip's bore 28. The alignment key 80 is sized and shaped to be closely received into a rounded slot 82 formed in the base 76 to align the grip 16—and hence the splint 12—in a desired and repeatable position when positioned on the base 76. The base 76 provides stability to the splint 12 during the process of advancing the patient's hand onto the grip 16. To do so, the base 76 is placed on a stable flat surface such as a tabletop. In some cases, depending on the needs of a given patient, the base may be secured to such a flat surface (e.g., a tabletop) by use of, for example, a clamp or other such mechanical device (not shown).

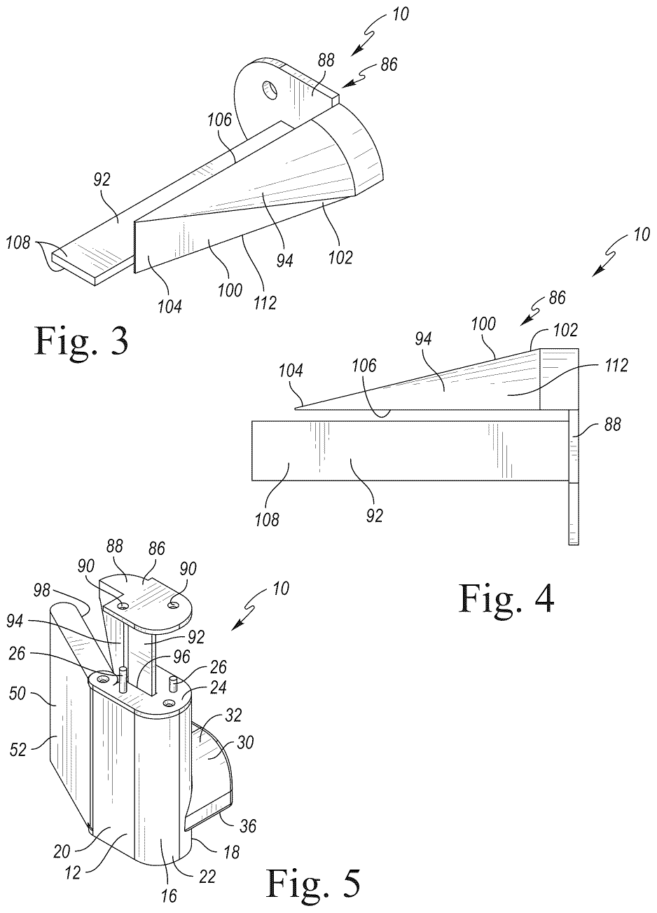

Referring now to FIGS. 3-5, the therapeutic device 10 also includes a deployment tool 86. The deployment tool 86 includes a proximal flange 88 that conforms to the size and shape of the proximal end 24 of the splint 12. As can be seen best in FIG. 6, the proximal flange 88 has a pair of alignment holes 90 that are sized and positioned to be received over the splint's mounting pins 26. The deployment tool 86 also includes an elongated key 92 and an elongated cam 94, both of which extend distally away from the bottom side of the proximal flange 88. In the illustrative embodiment described herein, the elongated key 92 is embodied as an elongated rectangular-shaped flange that is sized and shaped to be received into an elongated rectangular-shaped slot 96 formed in the splint's grip 16.

The elongated cam 94 is configured to engage a medial side 98 of the finger support flange 50 when the key 92 is positioned in the slot 96 of the grip 16 thereby moving the finger support flange 50 from its retracted position to its extended position. As can be seen in FIGS. 3 and 4, the elongated cam 94 includes a cam surface 100 that tapers downwardly from its proximal end 102 to its distal end 104 and a planar surface 106 opposite its cam surface 100. The elongated key 92 includes planar medial and lateral surfaces 108, both of which are perpendicular to the planar surface 106 of the elongated cam 94. When the key 92 is advanced into the grip's slot 96, the thin distal end 104 of the cam 94 wedges between the medial side 98 of the finger support flange 50 and the flat surface 110 of the grip 16 opposite its rounded surface 22. As the key 92 continues to be advanced downwardly into the grip's slot 96, the cam surface 100 of the cam 94 asserts a cam force on the medial side 98 of the finger support flange 50 thereby pivoting or otherwise rotating the finger support flange 50 relative to the grip 16. Fully seating the deployment tool's proximal flange 88 onto the proximal end 24 of the splint 12 positions the finger support flange 50 in its extended position. When so positioned, a planar lateral side 112 of the cam 94 acts as a stop to prevent movement of the finger support flange 50 back towards its retracted position.

In the illustrative embodiment described herein, the cam surface 100 of the cam 94 is configured to move the finger support flange 50 through a full 90-degrees of movement such that when positioned in its extended position, the lateral side 52 of the finger support flange 50 is aligned with (i.e., parallel with) the palm side 20 of the grip 16. However, in the case of treatment of patients who may not have the ability for such full extension of the fingers, other cam profiles may be used. For example, the cam surface 100 of the cam 94 may be configured to move the finger support flange 50 through 30-degrees of movement such that when positioned in its extended position, the lateral side 52 of the finger support flange 50 defines an obtuse angle of 150-degrees with the palm side 20 of the grip 16. In another example, the cam surface 100 of the cam 94 may be configured to move the finger support flange 50 through 60-degrees of movement such that when positioned in its extended position, the lateral side 52 of the finger support flange 50 defines an obtuse angle of 120-degrees with the palm side 20 of the grip 16. Other cam profiles producing other degrees of movement of the finger support flange 50 may be used to fit the needs of a given patient.

In use, the therapeutic device 10 may be used by a patient to stretch (i.e., extend) the patient's fingers and thumb as part of a regular therapeutic process in the treatment of spastic hemiparesis of the hand. To begin the process, the base 76 is placed on a stable flat surface such as a tabletop. In some cases, depending on the needs of a given patient, the base 76 may be secured to such a flat surface (e.g., a tabletop) by use of, for example, a clamp or other such mechanical device (not shown). If not already done so, the splint 12—with the install component 14 secured thereto—is positioned on the base 76 by positioning the base's post 78 in the splint's bore 28.

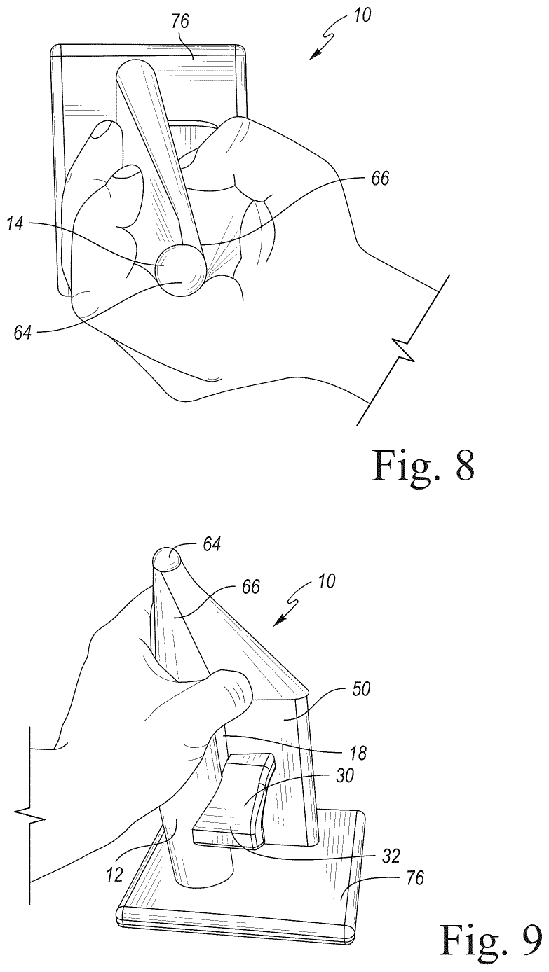

The patient then aligns the patient's thumb with the medial side of the install component 14 and the patient's fingers with the lateral side of the install component 14. The patient then moves the patient's hand downwardly such that the rounded proximal tip 64 is received between the patient's thumb and fingers. As shown in FIGS. 8 and 9, the patient then gradually advances the patient's hand downwardly (i.e., distally) along the conically-shaped guide body 66. The expanding outward taper of the conically-shaped guide body 66 causes the patient's fingers and thumb to spread apart thereby unclenching the patient's fisted hand.

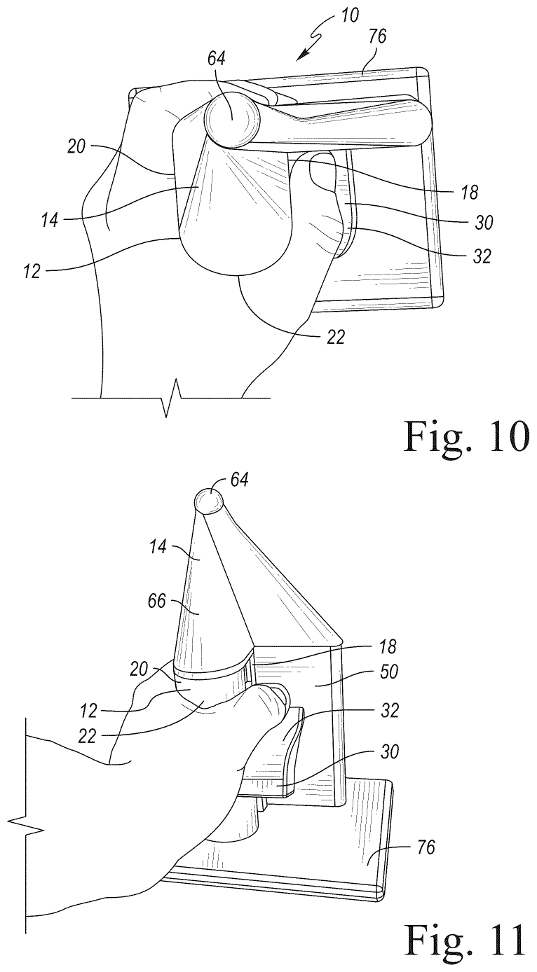

Continued downward advancement of the patient's hand causes the patient's thumb to transition off the surface of the install component 14 and into contact with the thumb side 18 of the splint's grip 16. Similarly, such continued downward advancement of the patient's hand causes the patient's fingers to transition off the surface of the install component 14 and into contact with the palm side 20 of the splint's grip 16. As shown in FIGS. 10 and 11, the patient continues to gradually advance the patient's hand downwardly until the patient's thumb comes to rest on the arcuate-shaped support surface 32 of the thumb support 30.

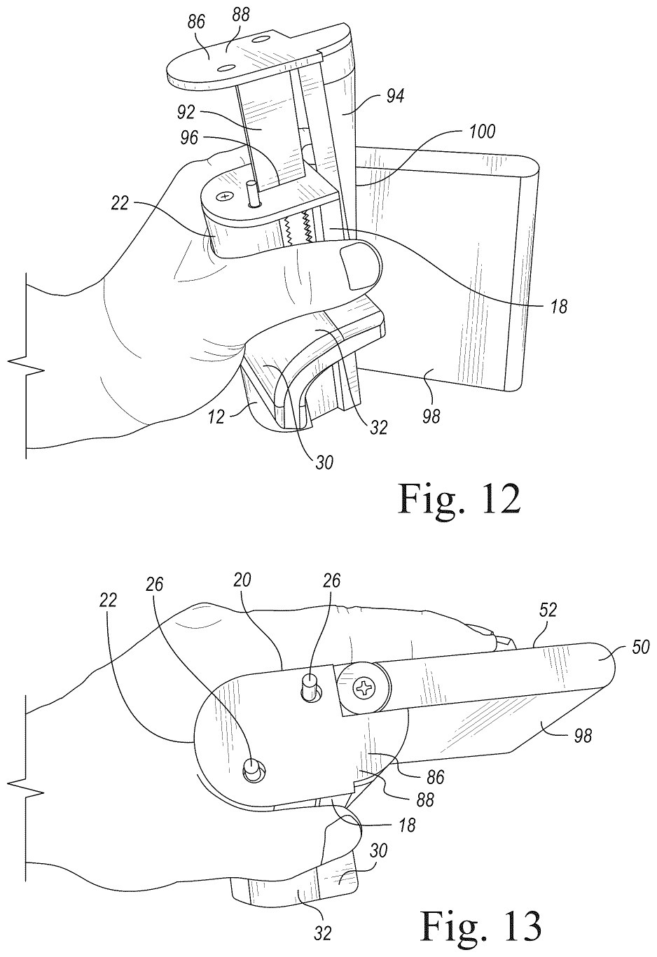

Thereafter, the patient removes the install component 14 from the splint 12. Once the install component 14 is removed, the patient may then use the deployment tool 86 to extend (e.g., straighten) the patient's fingers by moving the finger support flange 50 from its retracted position to its extended position, as shown in FIGS. 12 and 13. In the illustrative embodiment described herein, the patient uses the deployment tool 86 with the splint 12 removed from the base 76. However, in other embodiments, the patient may leave the splint 12 on the base 76 during use of the deployment tool 86. In either case, to use the deployment tool 86, the patient advances the key 92 into the grip's slot 96 thereby causing the thin distal end 104 of the cam 94 to wedge between the medial side 98 of the finger support flange 50 and the flat surface 110 of the grip 16 opposite its rounded surface 22. As the patient advances the key 92 downwardly into the grip's slot 96, the cam surface 100 of the cam 94 asserts a cam force on the medial side 98 of the finger support flange 50 thereby pivoting or otherwise rotating the finger support flange 50 relative to grip 16. As can be seen in FIG. 13, when the patient fully seats the deployment tool's proximal flange 88 onto the proximal end 24 of the splint 12, the finger support flange 50 is positioned in its extended position. When so positioned, the planar lateral side 112 of the cam 94 acts as a stop to prevent movement of the finger support flange 50 back towards it retracted position. As discussed above, the cam 94 may be embodied with cam profiles that cause less than 90-degrees of movement to full extension to fit the needs of a given patient.

Once the deployment tool 86 has been fully installed, the patient may then wear the splint 12 for the amount of time designated by the patient's treatment plan. Because the configuration of the splint 12 causes the patient to naturally grip it during treatment (as opposed to prior designs which require the hand to be flat), the patient may wear the splint 12 without the need for straps or other retainers. It should be appreciated, however, that in some cases a loose fitting strap may be used around the patient's metacarpophalangeal joints. Such a strap would not serve a therapeutic purpose, but would instead function as a retainer if a patient were to experience such a large finger stretch that the hand would lose the contracting force necessary to maintain a grip on the splint 12.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A therapeutic device for use in the treatment of spastic hemiparesis of the hand, comprising:
a splint having (i) a grip that includes a planar thumb side and an opposite planar palm side with a rounded surface connecting the thumb side to the palm side, (ii) a thumb support extending medially out of the thumb side of the grip, and (iii) a finger support flange pivotally coupled to the grip, and
an install component for guiding a hand of a patient onto the splint, the install component is removably secured to a proximal end of the splint and comprises (i) a rounded proximal tip, and (ii) a generally conically-shaped guide body extending distally away from the tip, the conically-shaped guide body tapers outwardly in both the mediolateral direction and the anteroposterior direction from its proximal end to its distal end.

2. The therapeutic device of claim 1, further comprising a base having (i) a platform, and (ii) a post extending upwardly from the base, wherein the post is sized and shaped to be received into a bore formed in the grip so as to allow the grip to be removably secured to the base.

3. The therapeutic device of claim 1, wherein the thumb support comprises an arcuate-shaped support surface.

4. The therapeutic device of claim 3, wherein the arcuate-shaped support surface is curved downwardly from its proximal end to its distal end.

5. The therapeutic device of claim 3, wherein the arcuate-shaped support surface is curved in the anteroposterior direction.

6. The therapeutic device of claim 1, wherein the thumb support is upwardly and downwardly adjustable relative to the grip.

7. The therapeutic device of claim 1, wherein:
the finger support flange is movable between an extended position and a retracted position,
a lateral side of the finger support flange is aligned with the palm side of the grip when the finger support flange is positioned in its extended position, and
the lateral side of the finger support flange is positioned perpendicular to the palm side of the grip when the finger support flange is positioned in its retracted position.

8. The therapeutic device of claim 7, further comprising a deployment tool having (i) an elongated key and (ii) an elongated cam, wherein:
the elongated key is sized and shaped to be received into an elongated slot formed in the grip, and
the elongated cam is configured to engage a medial side of the finger support flange when the key is positioned in the slot of the grip so as to move the finger support flange from its retracted position to its extended position.

9. The therapeutic device of claim 8, wherein:
the elongated cam comprises (i) a cam surface which tapers downwardly from its proximal end to its distal end, and (ii) a planar surface opposite its cam surface,
the elongated key includes planar medial and lateral surfaces, and
the planar surface of the elongated cam is perpendicular to the medial and lateral surfaces of the elongated key.

10. The therapeutic device of claim 1, wherein:
the finger support flange is movable between an extended position and a retracted position,
a lateral side of the finger support flange is positioned perpendicular to the palm side of the grip when the finger support flange is positioned in its retracted position, and
the lateral side of the finger support flange defines an obtuse angle with the palm side of the grip when the finger support flange is positioned in its extended position.

11. The therapeutic device of claim 10, further comprising a deployment tool having (i) an elongated key and (ii) an elongated cam, wherein:
the elongated key is sized and shaped to be received into an elongated slot formed in the grip, and
the elongated cam is configured to engage a medial side of the finger support flange when the key is positioned in the slot of the grip so as to move the finger support flange from its retracted position to its extended position.

12. A method of using a therapeutic device in the treatment of spastic hemiparesis of the hand, comprising:
positioning an install component on a proximal end of a splint,
advancing a patient's hand distally along a conically-shaped guide body of the install component and onto a grip of the splint, the conically-shaped guide body tapering outwardly in both the mediolateral direction and the anteroposterior direction from its proximal end to its distal end, and
pivoting, with the patient's hand positioned on the splint, a finger support flange of the splint from a retracted position to an extended position so as to extend one or more fingers of the patient's hand.

13. The method of claim 12, wherein during advancement of the patient's hand onto the grip of the splint (i) a thumb of the patient's hand is advanced into contact with a thumb side of the grip, and (ii) a palm of the patient's hand is advanced into contact with an opposite palm side of the grip.

14. The method of claim 12, wherein during advancement of the patient's hand onto the grip, a thumb of the patient's hand is advanced onto a thumb support extending medially out of the grip.

15. The method of claim 12, further comprising positioning the grip on a base prior to advancement of the patient's hand onto the grip of the splint.

16. The method of claim 15, further comprising removing the grip of the splint from the base prior to pivoting the finger support flange.

17. The method of claim 12, wherein pivoting the finger support flange comprises inserting a deployment tool into the grip so as to pivot the finger support flange from its retracted position to its extended position.

18. The method of claim 17, wherein inserting the deployment tool into the grip comprises inserting an elongated key of the deployment tool into the grip so as to cause an elongated cam of the deployment tool to pivot the finger support flange from its retracted position to its extended position.

19. A splint for use in the treatment of spastic hemiparesis of the hand, comprising:
a grip having a planar thumb side and an opposite planar palm side with a rounded surface connecting the thumb side to the palm side,
a thumb support extending medially out of the thumb side of the grip, the thumb support having an arcuate-shaped support surface, and
a finger support flange pivotally coupled to the grip.

20. The splint of claim 19, wherein the arcuate-shaped support surface is curved downwardly from its proximal end to its distal end.

21. The splint of claim 19, wherein the arcuate-shaped support surface is curved in the anteroposterior direction.

22. The splint of claim 19, wherein the thumb support is upwardly and downwardly adjustable relative to the grip.

23. The splint of claim 19, wherein:
the finger support flange is movable between an extended position and a retracted position,
a lateral side of the finger support flange is aligned with the palm side of the grip when the finger support flange is positioned in its extended position, and
the lateral side of the finger support flange is positioned perpendicular to the palm side of the grip when the finger support flange is positioned in its retracted position.

24. The splint of claim 19, wherein:
the finger support flange is movable between an extended position and a retracted position,
a lateral side of the finger support flange is positioned perpendicular to the palm side of the grip when the finger support flange is positioned in its retracted position, and
the lateral side of the finger support flange defines an obtuse angle with the palm side of the grip when the finger support flange is positioned in its extended position.
